(19) United States
(12) Patent Application Publication
(10) Pub. No.: US 2022/0178927 A1
(43) Pub. Date: Jun. 9, 2022

(54) BIOMARKERS FOR SELINEXOR

(71) Applicant: Karyopharm Therapeutics Inc., Newton, MA (US)

(72) Inventors: Andrea Califano, New York, NY (US); Mariano Javier Alvarez, El Cajon, CA (US); Yao Shen, Fort Lee, NJ (US)

(21) Appl. No.: 17/598,101

(22) PCT Filed: Mar. 27, 2020
(86) PCT No.: PCT/US20/25275

§ 371 Date: Sep. 24, 2021

Related U.S. Application Data
(60) Provisional application No. 62/824,877, filed on Mar. 27, 2019.

(51) Int. Cl. G01N 33/574; A61K 31/497; A61K 31/573; A61K 31/69; A61K 45/06; A61P 35/00; G16B 40/00; G16H 20/10

(52) U.S. Cl. CPC G01N 33/57426; A61K 31/497; A61K 31/573; A61K 31/69; C12Q 2600/158; A61P 35/00; G16B 40/00; G16H 20/10; G01N 2800/52; A61K 45/06

(57) ABSTRACT

A method of treating a patient suffering from multiple myeloma, comprising determining a plurality of protein activity values in a subject suffering from multiple myeloma (MM), each protein activity value corresponding to one of a set of proteins in the subject; determining a classification of the subject as a responder or non-responder to a therapy by a compound represented by structural formula (1); and administering a therapeutically effective amount of the compound represented by structural formula (1) or a pharmaceutically acceptable salt thereof.

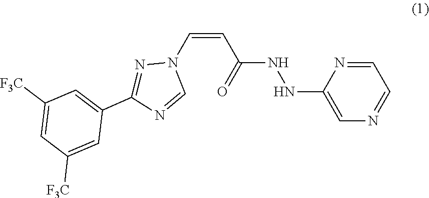 [SMILES: O=C(/C=C\n1cnc(-c2cc(C(F)(F)F)cc(C(F)(F)F)c2)n1)NNc1cnccn1]

### RELATED APPLICATION

This application claims the benefit of U.S. Provisional Application No. 62/824,877, filed on Mar. 27, 2019. The entire teachings of the above applications are incorporated herein by reference.

### BACKGROUND OF THE INVENTION

Multiple Myeloma (MM) is a hematological malignancy characterized by the accumulation of monoclonal plasma cells in the bone marrow, the presence of monoclonal immunoglobulin, or M protein in the serum or urine, bone disease, kidney disease, and immunodeficiency. MM is the second most common hematological malignancy (after non-Hodgkin's lymphoma), representing 1% of all cancers and 2% of all cancer deaths. The treatment of MM has improved in the last 20 years due to the use of high-dose chemotherapy and autologous stem cell transplantation, the introduction of immunomodulatory agents, such as thalidomide, lenalidomide, and pomalidomide, and the proteasome inhibitors, bortesomib and carfilzomib. However, despite the increased effectiveness of these agents, most patients develop resistant MM and succumb to the disease. As such, there remains a high unmet need to develop anti-MM agents and to tailor anti-MM therapies more closely to patients to achieve a higher likelihood of response.

### SUMMARY OF THE INVENTION

In an example embodiment, the present invention is a method of treating a patient suffering from multiple myeloma, comprising determining a plurality of protein activity values in the subject suffering from multiple myeloma (MM), each protein activity value corresponding to one of a set of proteins in the subject; determining a classification of the subject as a responder or non-responder to a therapy by a compound represented by structural formula (1); and administering a therapeutically effective amount of the compound represented by structural formula (1) or a pharmaceutically acceptable salt thereof

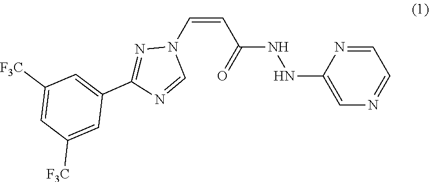 [SMILES: O=C(/C=C\n1cnc(-c2cc(C(F)(F)F)cc(C(F)(F)F)c2)n1)NNc1cnccn1]

to the subject determined to be responder.

In another example embodiment, the present invention is a method of treating a subject suffering from multiple myeloma, comprising administering a therapeutically effective amount of a compound represented by structural formula (1) or a pharmaceutically acceptable salt thereof

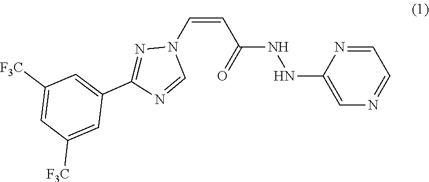 [SMILES: O=C(/C=C\n1cnc(-c2cc(C(F)(F)F)cc(C(F)(F)F)c2)n1)NNc1cnccn1]

to the subject suffering from multiple myeloma, wherein the subject is determined to be a responder to a therapy by the compound represented by structural formula (1) based on a plurality of protein activity values in the subject, each protein activity value corresponding to one of a set of proteins in the subject.

In another example embodiment, the present invention is a method of treating a subject suffering from multiple myeloma, comprising selecting the subject suffering from multiple myeloma only if the subject is determined to be a responder to a therapy by a compound represented by structural formula (1) based on a plurality of protein activity values in the subject, each protein activity value corresponding to one of a set of proteins in the subject; and administering to the selected subject a therapeutically effective amount of the compound represented by structural formula (1) or a pharmaceutically acceptable salt thereof

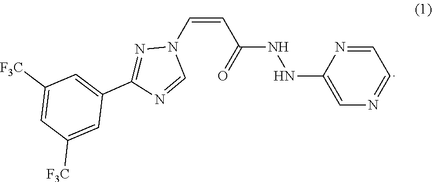 [SMILES: O=C(/C=C\n1cnc(-c2cc(C(F)(F)F)cc(C(F)(F)F)c2)n1)NNc1cnccn1]

In another example embodiment, the present invention is a method of treating a subject suffering from multiple myeloma, comprising receiving information of a plurality of protein activity values in a subject suffering from multiple myeloma (MM), each protein activity value corresponding to one of a set of proteins in the subject; and administering to the subject a therapeutically effective amount of a compound represented by structural formula (1) or a pharmaceutically acceptable salt thereof

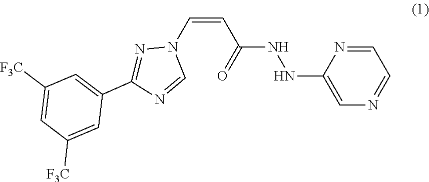 [SMILES: O=C(/C=C\n1cnc(-c2cc(C(F)(F)F)cc(C(F)(F)F)c2)n1)NNc1cnccn1]

only if the subject is determined to be a responder to a therapy by the compound represented by structural formula (1) based on said plurality of protein activity values.

In another example embodiment, the present invention is a method of identifying a subject as a responder or a non-responder, comprising determining a plurality of protein activity values in a subject suffering from multiple myeloma (MM), each protein activity value corresponding to one of a set of proteins in the subject; providing the plurality of protein activity values to a trained classifier, the trained classifier being trained to differentiate between responders and non-responders to a therapy by a compound represented by structural formula (1) or a pharmaceutically acceptable salt thereof; and obtaining from the classifier a classification of the subject as a responder or non-responder,

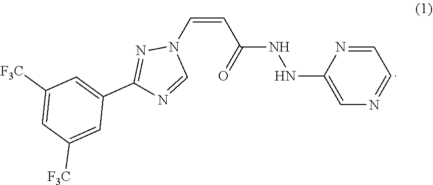 [SMILES: O=C(/C=C\n1cnc(-c2cc(C(F)(F)F)cc(C(F)(F)F)c2)n1)NNc1cnccn1]

In another example embodiment, the present invention is a computer program product for identifying responders and non-responders, the computer program product comprising a computer readable storage medium having program instructions embodied therewith, the program instructions executable by a processor to cause the processor to perform a method comprising determining a plurality of protein activity values in a subject suffering from multiple myeloma (MM), each protein activity value corresponding to one of a set of proteins in the subject; providing the plurality of protein activity values to a trained classifier, the trained classifier being trained to differentiate between responders and non-responders to a therapy by a compound represented by structural formula (1); and obtaining from the classifier a classification of the subject as a responder or non-responder,

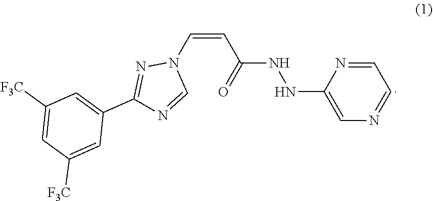 [SMILES: O=C(/C=C\n1cnc(-c2cc(C(F)(F)F)cc(C(F)(F)F)c2)n1)NNc1cnccn1]

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A and 1B illustrate analysis of 29 available interactomes based on tissue lineage supervised classification and network representation. Identification of the most appropriate tissue context-specific interactomes for MM was based on the likelihood predicted by a tissue-type classifier based on gene expression (FIG. 1A), and the Network Score (FIG. 1B), representing how well each evaluated interactome can explain the transcriptional state of the MM samples.

FIGS. 2A and 2B illustrate unsupervised hierarchical cluster analysis of protein activity signatures of responders and non-responders. FIG. 2A and FIG. 2B represents dendrograms showing the unsupervised clustering of the samples based on their similarity in Master Regulator (MR) signatures for patients that responded (FIG. 2A) and did not respond (FIG. 2B) to selinexor.

FIGS. 3A and 3B present data that demonstrate clinical benefits of the biomarkers for MM patients being treated with solinexor. FIG. 3A is heatmap showing the relative protein activity for the four example MR proteins. The bar above the heatmap shows the silhouette score for each sample, computed based on Euclidean distance, with Responder and Non-responder samples shown on the left and on the right, respectively. The values inside each cell in the heatmap shows the relative protein activity for each MR protein in each sample. FIG. 3B is a plot showing the evaluation of the biomarker performance in an independent sample set. Shown is the ROC analysis and estimated AUC.

FIG. 4 is a schematic of an example of a computing node

### DETAILED DESCRIPTION OF THE INVENTION

A description of example embodiments of the invention follows.

Targeting exportin 1 (XPO1) is a promising therapeutic option for patients with multiple myeloma (MM). Selinexor, a compound represented by the following structural formula,

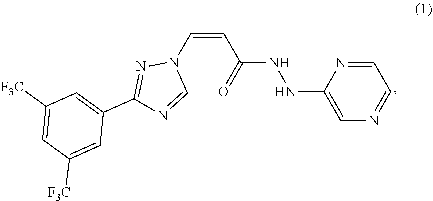 [SMILES: O=C(/C=C\n1cnc(-c2cc(C(F)(F)F)cc(C(F)(F)F)c2)n1)NNc1cnccn1]

in combination with low-dose dexamethasone, results in clinically meaningful responses for patients with MM refractory to currently available therapies.

A biomarker predictive of response was sought in MM patients treated with selinexor using the VIPER algorithm which can transform gene expression profiles from tumor samples into accurate predictions of protein activity for about 6,000 regulatory proteins. RNA levels in CD138+ cells isolated from the pre-treatment bone marrow aspirate of patients in STORM Part 2 clinical study were used to populate the VIPER algorithm.

The VIPER algorithm is described, for example, in WO2017/040311A1, the entire teachings of which are incorporated herein by reference.

Biomarkers predictive of response were identified. A linear discriminant analysis classifier trained on 35 pretreatment patient samples, including 16 responders and 19 non-responders, identified the following set of four proteins out of a larger set of approximately 100 proteins having protein predictive of a response to Selinexor (so called “Master Regulator” proteins, “MR”): IRF3, ARL2BP, ZBTB17, ATRX. These four MR proteins produced optimal predictive performance based on leave 1 out cross-validation (area under receiver operating characteristic curve (AUC)=0.862, P<0.01 by permutation testing). The 4-protein classifier was then validated on an independent, blinded 12-sample cohort of MM patients from STORM (Parts 1 and 2), achieving an AUC=0.77 (P≈0.06 by permutation analysis). Specifically, 4 of 5 responders and 6 of 7 non-responders to selinexor were correctly identified by the marker, yielding a prediction accuracy of 83%. Training the classifier using differential gene expression data alone produced no statistically significant classification.

Additional MR proteins are shown in Table 1. The top 100 proteins showing differential activity between responder and non-responder patients are given. The first four of this list are described further above. The third column indicates the False Discovery Rate (FDR)-corrected p-value. Statistical significance for the differential activity of regulatory proteins was estimated by the Student t-test, and p-values were corrected to account for multiple hypothesis tests using the False Discovery Rate (FDR) method according to Benjamini & Hochberg (Benjamini, Y., and Hochberg, Y. (1995). Controlling the false discovery rate: a practical and powerful approach to multiple testing. Journal of the Royal Statistical Society Series B, 57, 289-300. http://www.jstor.org/stable/2346101), the entire teachings of which is hereby incorporated by reference.

### Biomarkers for Response in Multiple Myeloma Patients

In an example embodiment, the following four MR proteins can be used as biomarkers of Selinexor response in MM patients.

Human IRF3, Interferon Regulatory Protein 3, is described, for example, as UniProtKB: Q14653 at the URL https://www.uniprot.org/uniprot/Q14653.

Human ARL2BP, ADP-ribosylation factor-like protein 2-binding protein, is described, for example, as UniProtKB Q9Y2Y0 at the URL https://www.uniprot.org/uniprot/Q9Y2Y0.

Human ZBTB17, Zinc finger and BTB domain-containing protein 17, is described, for example, as UniProtKB Q13105 at the URL https://www.uniprot.org/uniprot/Q13105.

Human ATRX, Transcriptional regulator ATRX, is described, for example, as UniProtKB P46100, at the URL https://www.uniprot.org/uniprot/P46100.

### Determining Protein Activity

In various embodiments, protein activity is determined for one or more subjects based on genetic data. Protein activity for a population of subjects is used to identify MR proteins as described above, and to train classifiers based on sets of known responders and non-responders. Similarly, protein activity for an individual subject is used to classify that subject as a responder or non-responder. In particular, a feature vector is constructed for a given subject that comprises protein activity values for one or more proteins.

Various measures of protein activity are suitable for use according to the present disclosure. For example, as described further below, VIPER provides protein activity values in terms of normalized enrichment scores, which express activity for all the regulatory proteins in the same scale. However, it will be appreciated that alternative methods of determining protein activity provide alternative measures of protein activity values, for example, absolute or relative abundance in a sample, or absolute enrichment.

Various embodiments described herein employ the VIPER algorithm to determine protein activity in the form of normalized enrichment scores for a plurality of proteins based on a predetermined model of transcriptional regulation. The VIPER algorithm is described further in PCT Pub. No. WO2017040311A1, which is hereby incorporated by reference in its entirety.

It will be appreciated that alternative methods of determining protein activity in a subject are also applicable for practicing the methods described herein. Exemplary alternative algorithms for inferring protein activity from gene expression data include: ChIP-X Enrichment Analysis (ChEA), which is described further in Keenan, A. B. et al. ChEA3: transcription factor enrichment analysis by orthogonal omics integration. Nucleic Acids Res. 47, W212-W224 (2019); TFEA.ChIP, which is described further in Puente-Santamaria, L., Wasserman, W. W. & Del Peso, L. TFEA.ChIP: a tool kit for transcription factor binding site enrichment analysis capitalizing on ChIP-seq datasets. Bioinformatics 35, 5339-5340 (2019); Binding Analysis for Regulation of Transcription (BART), which is described further in Wang, Z. et al. BART: a transcription factor prediction tool with query gene sets or epigenomic profiles. Bioinformatics 34, 2867-2869 (2018); Mining Gene Cohorts for Transcriptional Regulators Inferred by Kolmogorov-Smirnov Statistics (MAGICTRICKS), which is described further in Roopra A. MAGICTRICKS: A tool for predicting transcription factors and cofactors that drive gene lists. https://doi.org/10.1101/492744; DoRothEA, which is described further in Garcia-Alonso, L. et al. Transcription factor activities enhance markers of drug sensitivity in cancer. Cancer Res. 78, 769-780 (2018); and NetFactor, which is described further in Ahsen, M. E. et al. NeTFactor, a framework for identifying transcriptional regulators of gene expression-based biomarkers. Sci. Rep. 9, 12970 (2019).

In addition, biochemical approaches can be used to estimate abundance of the proteins included in a given biomarker, such us immunostaining (immunofluorescence or immunochemistry) of tissue samples followed by histological examination, flow cytometry, mass cytometry or cytometric bead arrays, reverse-phase protein arrays, bead-based IVD assays such as Luminex and mass spectrometry.

### Classification of Subjects

A set of MR proteins may be determined by a variety of methods, including those described in connection with the examples below. For example, cluster analysis may be performed with or without separate dimensionality reduction in order to determine the heterogeneity of responder and non-responder clusters in an n-dimensional vector space, with n corresponding to a number of proteins considered. It will be appreciated that a variety of methods are available for dimensionality reduction, including unsupervised dimensionality reduction techniques such as principal component analysis (PCA), random projection, and feature agglomeration analysis. It will further be appreciated that a variety of cluster analysis methods are available, including hierarchical clustering and k-means clustering. It will be appreciated that a variety of statistical methods are available for determining the correlation of a given protein value to the classification as a responder or non-responder.

In various embodiments described, the DarwinOncoTarget™ system is used to identify and rank potential protein predictors of responsiveness and non-responsiveness. Table 1 provides a listing of the top 100 proteins showing differential activity between responder and non-responder patients, sorted by the False Discovery Rate (FDR)-corrected p-value. The first four of this list provide the exemplary biomarker described herein.

In various embodiments, a subset of proteins is selected by performing a cross-validation process such as leave-one-out cross validation. In such embodiments, a model is trained on all data except for one point and a prediction is made for that point. It will be appreciated that cross-validation may be used to optimize the selection of proteins and/or the number of proteins. In addition, repeated application of cross-validation may be employed with multiple models in order to select an optimal pairing of model and proteins. Accordingly, it will be appreciated that a variable number of proteins may be selected for training a classifier as set out herein. In particular, in various embodiments, any subset of the MR proteins provided in Table 1 may be used to train one or more classifier. It will be appreciated that while there may be computational advantages to reduction in the number of MR proteins used to train a given classifier, a classifier may be trained with all or some of the potential proteins while still arriving at a trained classifier suitable for identification of responders and non-responders. In particular, while inclusion of additional low value proteins may increase training time, a given classifier will de-emphasize low value proteins while emphasizing high value proteins by virtue of the training process. In some embodiments, a predetermined number of proteins having the highest differential activity between responder and non-responder patients are selected.

A training set including responders and non-responders is determined by RNA sequencing of a plurality of subjects. Normalized enrichment scores (NES) are determined for a plurality of proteins across the training set. In some embodiments, normalized enrichment scores are determined by application of VIPER.

During a training phase according to various embodiments, protein activity scores for responsive and non-responsive subjects are determined as set forth above. A feature vector is constructed for each of the responsive and non-responsive subjects, and provided to a classifier. In some embodiments, the classifier comprises a SVM. In some embodiments, the classifier comprises an artificial neural network. In some embodiments, the classifier comprises a random decision forest. It will be appreciated that a variety of other classifiers are suitable for use according to the present disclosure, including linear classifiers, support vector machines (SVM), Linear Discriminant Analysis (LDA), Logistic regression, Random Forest, Ridge regression methods, or neural networks such as recurrent neural networks (RNN). In addition, it will be appreciated that an ensemble model of any of the forgoing may also be employed.

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Based upon the training set, the classifier is trained to classify a subject as either responsive or non-responsive.

In a classification phase according to various embodiments, a protein activity of a given subject is determined. The protein activity values are provided as a feature vector to a trained classifier, which provides an output classification as either a responder or a non-responder.

TABLE 1

| | | | |
|---|---|---|---|
| 3661 | IRF3 | 7.85E−19 | interferon regulatory factor 3 |
| 23568 | ARL2BP | 1.87E−18 | ADP-ribosylation factor-like 2 binding protein |
| 7709 | ZBTB17 | 2.79E−16 | zinc finger and BTB domain containing 17 |
| 546 | ATRX | 4.03E−16 | alpha thalassemia/mental retardation syndrome X-linked |
| 143098 | MPP7 | 5.93E−16 | membrane protein, palmitoylated 7 (MAGUK p55 |
| | | | subfamily member 7) |
| 51567 | TDP2 | 7.12E−16 | tyrosyl-DNA phosphodiesterase 2 |
| 466 | ATF1 | 1.11E−14 | activating transcription factor 1 |
| 23291 | FBXW11 | 2.19E−14 | F-box and WD repeat domain containing 11 |
| 10438 | C1D | 4.79E−14 | C1D nuclear receptor corepressor |
| 5310 | PKD1 | 1.45E−13 | polycystic kidney disease 1 (autosomal dominant) |
| 2665 | GDI2 | 4.54E−13 | GDP dissociation inhibitor 2 |
| 6829 | SUPT5H | 7.01E−13 | suppressor of Ty 5 homolog (S. cerevisiae) |
| 8036 | SHOC2 | 9.79E−13 | soc-2 suppressor of clear homolog (C. elegans) |
| 10616 | RBCK1 | 3.21E−12 | RanBP-type and C3HC4-type zinc finger containing 1 |
| 90850 | ZNF598 | 1.05E−11 | zinc finger protein 598 |
| 90874 | ZNF697 | 3.54E−11 | zinc finger protein 697 |
| 5567 | PRKACB | 6.95E−11 | protein kinase, cAMP-dependent, catalytic, beta |
| 51547 | SIRT7 | 6.95E−11 | sirtuin 7 |
| 6198 | RPS6KB1 | 1.97E−10 | ribosomal protein S6 kinase, 70 kDa, polypeptide 1 |
| 5861 | RAB1A | 2.41E−10 | RAB1A, member RAS oncogene family |
| 284346 | ZNF575 | 2.84E−10 | zinc finger protein 575 |
| 54799 | MBTD1 | 3.32E−10 | mbt domain containing 1 |
| 7572 | ZNF24 | 3.32E−10 | zinc finger protein 24 |
| 10607 | TBL3 | 3.76E−10 | transducin (beta)-like 3 |
| 10514 | MYBBP1A | 4.38E−10 | MYB binding protein (P160) 1a |
| 9620 | CELSR1 | 5.78E−10 | cadherin, EGF LAG seven-pass G-type receptor 1 |
| | | | (flamingo homolog, Drosophila) |
| 9739 | SETD1A | 6.04E−10 | SET domain containing 1A |
| 7157 | TP53 | 1.38E−09 | tumor protein p53 |
| 9994 | CASP8AP2 | 1.38E−09 | caspase 8 associated protein 2 |
| 7576 | ZNF28 | 1.86E−09 | zinc finger protein 28 |
| 6794 | STK11 | 2.31E−09 | serine/threonine kinase 11 |
| 6597 | SMARCA4 | 3.15E−09 | SWI/SNF related, matrix associated, actin dependent |
| | | | regulator of chromatin, subfamily a, member 4 |
| 23411 | SIRT1 | 3.27E−09 | sirtuin 1 |
| 388569 | ZNF324B | 3.35E−09 | zinc finger protein 324B |
| 55205 | ZNF532 | 5.40E−09 | zinc finger protein 532 |
| 53615 | MBD3 | 6.60E−09 | methyl-CpG binding domain protein 3 |
| 9765 | ZFYVE16 | 1.10E−08 | zinc finger, FYVE domain containing 16 |
| 7812 | CSDE1 | 1.16E−08 | cold shock domain containing E1, RNA-binding |
| 11020 | IFT27 | 1.16E−08 | intraflagellar transport 27 homolog (Chlamydomonas) |
| 5187 | PER1 | 1.70E−08 | period homolog 1 (Drosophila) |
| 80204 | FBXO11 | 1.88E−08 | F-box protein 11 |
| 8804 | CREG1 | 1.88E−08 | cellular repressor of E1A-stimulated genes 1 |
| 9191 | DEDD | 2.30E−08 | death effector domain containing |
| 1855 | DVL1 | 2.52E−08 | dishevelled, dsh homolog 1 (Drosophila) |
| 54386 | TERF2IP | 2.52E−08 | telomeric repeat binding factor 2, interacting protein |
| 29066 | ZC3H7A | 5.19E−08 | zinc finger CCCH-type containing 7A |
| 7297 | TYK2 | 5.87E−08 | tyrosine kinase 2 |
| 1455 | CSNK1G2 | 1.04E−07 | casein kinase 1, gamma 2 |
| 949 | SCARB1 | 1.41E−07 | scavenger receptor class B, member 1 |
| 1877 | E4F1 | 1.64E−07 | E4F transcription factor 1 |
| 3281 | HSBP1 | 3.96E−07 | heat shock factor binding protein 1 |
| 84240 | ZCCHC9 | 4.07E−07 | zinc finger, CCHC domain containing 9 |
| 10295 | BCKDK | 4.88E−07 | branched chain ketoacid dehydrogenase kinase |
| 25865 | PRKD2 | 4.93E−07 | protein kinase D2 |
| 1059 | CENPB | 5.83E−07 | centromere protein B, 80 kDa |
| 55294 | FBXW7 | 1.03E−06 | F-box and WD repeat domain containing 7 |
| 146542 | ZNF688 | 1.33E−06 | zinc finger protein 688 |
| 7323 | UBE2D3 | 2.10E−06 | ubiquitin-conjugating enzyme E2D 3 (UBC4/5 homolog, yeast) |
| 59307 | SIGIRR | 2.67E−06 | single immunoglobulin and toll-interleukin 1 receptor |
| | | | (TIR) domain |
| 9641 | IKBKE | 2.82E−06 | inhibitor of kappa light polypeptide gene enhancer in |
| | | | B-cells, kinase epsilon |
| 81857 | MED25 | 2.95E−06 | mediator complex subunit 25 |
| 140460 | ASB7 | 2.95E−06 | ankyrin repeat and SOCS box containing 7 |
| 3020 | H3F3A | 3.21E−06 | H3 histone, family 3A |
| 23373 | CRTC1 | 3.64E−06 | CREB regulated transcription coactivator 1 |
| 84256 | FLYWCH1 | 5.02E−06 | FLYWCH-type zinc finger 1 |
| 25909 | AHCTF1 | 6.33E−06 | AT hook containing transcription factor 1 |
| 2101 | ESRRA | 6.53E−06 | estrogen-related receptor alpha |
| 4793 | NFKBIB | 7.78E−06 | nuclear factor of kappa light polypeptide gene |
| | | | enhancer in B-cells inhibitor, beta |
| 90317 | ZNF616 | 9.17E−06 | zinc finger protein 616 |
| 1018 | CDK3 | 9.70E−06 | cyclin-dependent kinase 3 |
| 23645 | PPP1R15A | 9.79E−06 | protein phosphatase 1, regulatory (inhibitor) subunit 15A |
| 84335 | AKT1S1 | 1.07E−05 | AKT1 substrate 1 (proline-rich) |
| 51742 | ARID4B | 1.88E−05 | AT rich interactive domain 4B (RBP1-like) |
| 23067 | SETD1B | 2.35E−05 | SET domain containing 1B |
| 30001 | ERO1L | 2.66E−05 | ERO1-like (S. cerevisiae) |
| 127428 | TCEANC2 | 3.05E−05 | transcription elongation factor A (SII) N-terminal and |
| | | | central domain containing 2 |
| 4296 | MAP3K11 | 3.25E−05 | mitogen-activated protein kinase kinase kinase 11 |
| 5699 | PSMB10 | 3.27E−05 | proteasome (prosome, macropain) subunit, beta type, 10 |
| 5589 | PRKCSH | 4.00E−05 | protein kinase C substrate 80K-H |
| 140467 | ZNF358 | 4.52E−05 | zinc finger protein 358 |
| 284443 | ZNF493 | 4.68E−05 | zinc finger protein 493 |
| 5494 | PPM1A | 5.73E−05 | protein phosphatase, Mg2+/Mn2+ dependent, 1A |
| 23162 | MAPK8IP3 | 5.95E−05 | mitogen-activated protein kinase 8 interacting protein 3 |
| 8690 | JRKL | 6.71E−05 | jerky homolog-like (mouse) |
| 10555 | AGPAT2 | 7.05E−05 | 1-acylglycerol-3-phosphate O-acyltransferase 2 |
| | | | (lysophosphatidic acid acyltransferase, beta) |
| 3006 | HIST1H1C | 7.16E−05 | histone cluster 1, H1c |
| 10163 | WASF2 | 7.70E−05 | WAS protein family, member 2 |
| 79697 | C14orf169 | 7.83E−05 | chromosome 14 open reading frame 169 |
| 54453 | RIN2 | 9.59E−05 | Ras and Rab interactor 2 |
| 8726 | EED | 0.000106 | embryonic ectoderm development |
| 163033 | ZNF579 | 0.000118 | zinc finger protein 579 |
| 286205 | SCAI | 0.000129 | suppressor of cancer cell invasion |
| 4605 | MYBL2 | 0.000139 | v-myb myeloblastosis viral oncogene homolog (avian)-like 2 |
| 11218 | DDX20 | 0.000151 | DEAD (Asp-Glu-Ala-Asp) box polypeptide 20 |
| 1201 | CLN3 | 0.000181 | ceroid-lipofuscinosis, neuronal 3 |
| 7290 | HIRA | 0.000183 | HIR histone cell cycle regulation defective homolog A |
| | | | (S. cerevisiae) |
| 55906 | ZC4H2 | 0.000197 | zinc finger, C4H2 domain containing |
| 9213 | XPR1 | 0.000214 | xenotropic and polytropic retrovirus receptor 1 |
| 22827 | PUF60 | 0.000228 | poly-U binding splicing factor 60 KDa |
| 3212 | HOXB2 | 0.000228 | homeobox B2 |

### Methods of Treating

In a first example embodiment, the present invention is a method of treating a patient suffering from multiple myeloma, comprising determining a plurality of protein activity values in the subject suffering from multiple myeloma (MM), each protein activity value corresponding to one of a set of proteins in the subject; determining a classification of the subject as a responder or non-responder to a therapy by a compound represented by structural formula (1); and administering a therapeutically effective amount of the compound represented by structural formula (1) or a pharmaceutically acceptable salt thereof

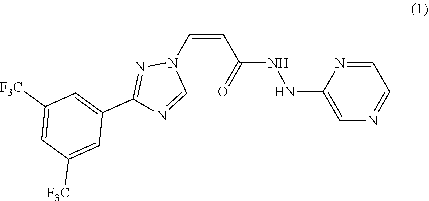 [SMILES: O=C(/C=C\n1cnc(-c2cc(C(F)(F)F)cc(C(F)(F)F)c2)n1)NNc1cnccn1]

to the subject determined to be responder.

In a second example embodiment, the present invention is a method of treating a subject suffering from multiple myeloma, comprising administering a therapeutically effective amount of a compound represented by structural formula (1) or a pharmaceutically acceptable salt thereof

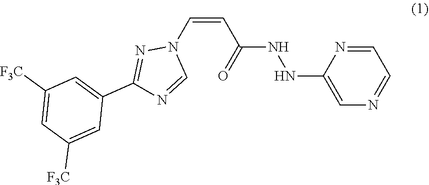 [SMILES: O=C(/C=C\n1cnc(-c2cc(C(F)(F)F)cc(C(F)(F)F)c2)n1)NNc1cnccn1]

to the subject suffering from multiple myeloma, wherein the subject is determined to be a responder to a therapy by the compound represented by structural formula (1) based on a plurality of protein activity values in the subject, each protein activity value corresponding to one of a set of proteins in the subject.

In a third example embodiment, the present invention is a method of treating a subject suffering from multiple myeloma, comprising selecting the subject suffering from multiple myeloma only if the subject is determined to be a responder to a therapy by a compound represented by structural formula (1) based on a plurality of protein activity values in the subject, each protein activity value corresponding to one of a set of proteins in the subject; and administering to the selected subject a therapeutically effective amount of the compound represented by structural formula (1) or a pharmaceutically acceptable salt thereof

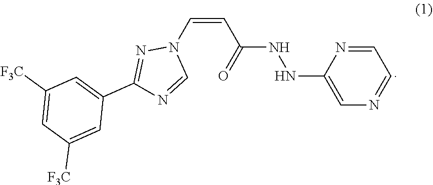 [SMILES: O=C(/C=C\n1cnc(-c2cc(C(F)(F)F)cc(C(F)(F)F)c2)n1)NNc1cnccn1]

In a fourth example embodiment, the present invention is a method of treating a subject suffering from multiple myeloma, comprising receiving information of a plurality of protein activity values in a subject suffering from multiple myeloma (MM), each protein activity value corresponding to one of a set of proteins in the subject; and administering to the subject a therapeutically effective amount of a compound represented by structural formula (1) or a pharmaceutically acceptable salt thereof

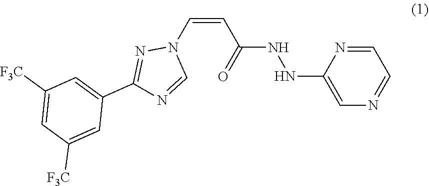 [SMILES: O=C(/C=C\n1cnc(-c2cc(C(F)(F)F)cc(C(F)(F)F)c2)n1)NNc1cnccn1]

only if the subject is determined to be a responder to a therapy by the compound represented by structural formula (1) based on said plurality of protein activity values.

In a first aspect of the first through fourth example embodiments, the set of proteins is selected from IRF3, ARL2BP, ZBTB17, ATRX, MPP7, TDP2, ATF1, FBXW11, C1D, PKD1, GDI2, SUPT5H, SHOC2, RBCK1, ZNF598, ZNF697, PRKACB, SIRT7, RPS6KB1, RAB1A, ZNF575, MBTD1, ZNF24, TBL3, MYBBP1A, CELSR1, SETD1A, TP53, CASP8AP2, ZNF28, STK11, SMARCA4, SIRT1, ZNF324B, ZNF532, MBD3, ZFYVE16, CSDE1, IFT27, PER1, FBXO11, CREG1, DEDD, DVL1, TERF2IP, ZC3H7A, TYK2, CSNK1G2, SCARB1, E4F1, HSBP1, ZCCHC9, BCKDK, PRKD2, CENPB, FBXW7, ZNF688, UBE2D3, SIGIRR, IKBKE, MED25, ASB7, H3F3A, CRTC1, FLYWCH1, AHCTF1, ESRRA, NFKBIB, ZNF616, CDK3, PPP1R15A, AKT1S1, ARID4B, SETD1B, ERO1L, TCEANC2, MAP3K11, PSMB10, PRKCSH, ZNF358, ZNF493, PPM1A, MAPK8IP3, JRKL, AGPAT2, HIST1H1C, WASF2, C14orf169, RIN2, EED, ZNF579, SCAI, MYBL2, DDX20, CLN3, HIRA, ZC4H2, XPR1, PUF60, and HOXB2.

In a second aspect of the first through fourth example embodiments, the set of proteins is IRF3, ARL2BP, ZBTB17, and ATRX.

In a third aspect of the first through fourth example embodiments and all aspects thereof, the method further comprises collecting a bone marrow sample from the subject; separating CD131+ cells in the bone marrow sample; and identifying the activity pattern of the MR proteins in the CD131+ cells.

In a fourth aspect of the first through fourth example embodiments and all aspects thereof, the multiple myeloma is a refractory multiple myeloma.

In a fifth aspect of the first through fourth example embodiments and all aspects thereof, the subject has received from 1 to 7 prior therapies, for example, the subject has received at least two prior therapies, or at least three prior therapies.

In a sixth aspect of the first through fourth example embodiments and all aspects thereof, the subject is an adult human.

In a seventh aspect of the first through fourth example embodiments and all aspects thereof, the multiple myeloma is relapsed or refractory multiple myeloma (RRMM).

In an eighth aspect of the first through fourth example embodiments and all aspects thereof, the subject has relapsed refractory multiple myeloma (RRMM) and has received at least four prior therapies.

In a ninth aspect of the first through fourth example embodiments and all aspects thereof, the subject has relapsed refractory multiple myeloma, has received at least four prior therapies and the relapsed or refractory multiple myeloma is refractory to at least two proteasome inhibitors, at least two immunomodulatory agents, and an anti-CD38 monoclonal antibody.

In a tenth aspect of the first through fourth example embodiments and all aspects thereof, the method of treating further includes administration of therapeutically effective amount of dexamethasone. In a particular aspect, the therapeutically effective amount of dexamethasone ranges from about 100 mg/day to about 10 mg/day. In a further particular aspect, the therapeutically effective amount of dexamethasone is 20 mg/day.

In an eleventh aspect of the first through fourth example embodiments and all aspects thereof, the method of treating comprises orally administering 80 mg of the compound represented by formula (1) and 20 mg of dexamethasone to an adult human subject on days 1 and 3 of each week of treatment, wherein the subject is suffering from relapsed refractory multiple myeloma, has received at least four prior therapies and further wherein the relapsed or refractory multiple myeloma is refractory to at least two proteasome inhibitors, at least two immunomodulatory agents, and an anti-CD38 monoclonal antibody. For example, if treatment is started on Tuesday and that is day 1 of treatment, then day 3 of treatment would be Thursday.

In an twelfth aspect of the first through fourth embodiments and all aspects thereof the method of treating comprises administering a compound of formula (1) in combination with at least one (e.g, 1, 2 or 3) of the following: lenalidomide, pomalidomide, carfilzomib, bortezomib or duratumumab and optionally dexamethasone. The combination administration of this embodiment can be twice a week (e.g., Days 1 and 3) or once per week. In one aspect, the patient receiving the combination therapy of the compound of formula (1), bortezomib and optionally dexamethasone has not been previously treated with a proteasome inhibitor (PI naïve).

As used above, “all aspects thereof” includes aspects numbered both before and after the given aspect.

A “therapeutically effective amount”, as used herein refers to an amount that is sufficient to achieve a desired therapeutic effect. For example, a therapeutically effective amount can refer to an amount that is sufficient to improve at least one sign or symptom of diseases or conditions disclosed herein. In a particular embodiment, the therapeutically effective amount of the compound of formula (1) is from about 200 mg to about 20 mg. In a further particular embodiment, the therapeutically effective amount of the compound of formula (1) is 80 mg per administration. In a particular dosing regimen, the compound of formula (1) is administered on Days 1 and 3 of each week of treatment at a dose of 80 mg per administration. In an even more particular embodiment, the compound of formula (1) is administered on Days 1 and 3 of each week of treatment at a dose of 80 mg per administration and 20 mg of dexamethasone is co-administered on the same days as the compound of formula (1). In a specific aspect of the dosing regimen, the compound of formula (1) and dexamethasone are administered orally.

In a further embodiment, the compound of formula (1) is administered once per week. In a particular aspect, the amount is from about 20 mg to about 200 mg. In a more particular aspect, the amount of the compound of formula (1) administered is about 80 mg.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. In particular, subjects are humans, such as adult humans. In one embodiment, the subject is an adult human. In a specific aspect, the adult human subject is suffering from relapsed refractory multiple myeloma. In a further aspect, the adult human subject has received at least four prior therapies to treat the relapsed refractory multiple myeloma. In yet a further aspect, the adult human subject has received at least four prior therapies to treat the relapsed refractory multiple myeloma and the relapsed refractory multiple myeloma is refractory to at least two proteasome inhibitors, at least two immunomodulatory agents, and an anti-CD38 monoclonal antibody.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition.

The phrase “combination therapy” or “co-administration” embraces the administration of the compound of Formula (I) and an additional therapeutic agent as part of a specific treatment regimen intended to provide a beneficial effect from the co-action of each. When administered as a combination, the compound of Formula (I) and an additional therapeutic agent can be formulated as separate compositions. Administration of these therapeutic agents in combination typically is carried out over a defined time period (usually minutes, hours, days or weeks depending upon the combination selected).

“Combination therapy” or “co-administration” is intended to embrace administration of these therapeutic agent (the compound of Formula (I) and an additional therapeutic agent) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence wherein the therapeutic agents are administered is not narrowly critical. “Combination therapy” also can embrace the administration of the therapeutic agents as described above in further combination with other biologically active ingredients (such as, but not limited to, a second and different therapeutic agent) and non-drug therapies (e.g., surgery or radiation). In a particular embodiment, dexamethasone is co-administered with the compound of formula (1). In an even more particular embodiment, the dexamethasone is administered at 20 mg per administration.

In another embodiment, combination treatment comprises the administration of the compound represented by formula (1) in combination with at least one (e.g., 1, 2 or 3) of the following: lenalidomide, pomalidomide, carfilzomib, bortezomib or duratumumab and optionally dexamethasone. The combination administration of this embodiment can be twice a week (e.g., Days 1 and 3) or once per week. In one aspect, the treatment comprises administering a combination of the compound of formula (1), bortezomib and optionally dexamethasone. In a particular aspect of this embodiment, the subject has not been previously treated with a proteasome inhibitor (PI naïve). In an example embodiment having a 35 day cycle, selinexor is administered on Days 1, 8, 15, 22, and 29 of a 35-day cycle (e.g., at 100 mg per dose); bortezomib is administered on Days 1, 8, 15, and 22 of a 35-day cycle (e.g., at 1.3 mg/m2) and dexamethasone is administered Days 1, 2, 8, 9, 15, 16, 22, 23, 29, and 30 of each 35-day cycle at 20 mg per dose. The length of the cycle can be adjusted accordingly, maintaining the once weekly administration for selinexor and bortezomib and the twice weekly administration of dexamethasone.

The compounds of formula (1) can be present in the form of pharmaceutically acceptable salt. For use in medicines, the salts of the compounds of formula (1) refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The compounds of formula (1) can be administered orally, nasally, ocularly, transdermally, topically, intravenously (both bolus and infusion), and via injection (intraperitoneally, subcutaneously, intramuscularly, intratumorally, or parenterally) either as alone or as part of a pharmaceutical composition comprising the compound of formula (1) and a pharmaceutically acceptable excipient. The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository.

In a particular embodiment, the compound of formula (1) and optionally a second agent (e.g., dexamethasone) is administered orally. Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions.

As used herein, prior therapies refers to known therapies for multiple myeloma involving administration of a therapeutic agent. Prior therapies can include, but are not limited to, treatment with proteasome inhibitors (PI), Immunomodulatory agents, anti-CD38 monoclonal antibodies or other agents typically used in the treatment of multiple myeloma such as glucocorticoids. Specific prior therapies can include bortezomib, carfilzomib, lenalidomide, pomalidomide, daratumumab, glucocorticoids or an alkylating agent.

### Methods of Classifying a Subject

In a fifth example embodiment, the present invention is a method of identifying a subject as a responder or a non-responder, comprising determining a plurality of protein activity values in a subject suffering from multiple myeloma (MM), each protein activity value corresponding to one of a set of proteins in the subject; providing the plurality of protein activity values to a trained classifier, the trained classifier being trained to differentiate between responders and non-responders to a therapy by a compound represented by structural formula (1); and obtaining from the classifier a classification of the subject as a responder or non-responder,

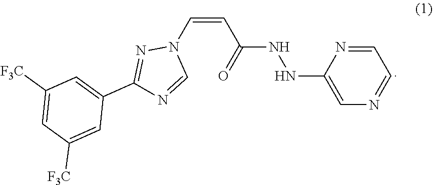 [SMILES: O=C(/C=C\n1cnc(-c2cc(C(F)(F)F)cc(C(F)(F)F)c2)n1)NNc1cnccn1]

In a first aspect of the fifth example embodiment, the set of proteins is selected from IRF3, ARL2BP, ZBTB17, ATRX, MPP7, TDP2, ATF1, FBXW11, C1D, PKD1, GDI2, SUPT5H, SHOC2, RBCK1, ZNF598, ZNF697, PRKACB, SIRT7, RPS6KB1, RAB1A, ZNF575, MBTD1, ZNF24, TBL3, MYBBP1A, CELSR1, SETD1A, TP53, CASP8AP2, ZNF28, STK11, SMARCA4, SIRT1, ZNF324B, ZNF532, MBD3, ZFYVE16, CSDE1, IFT27, PER1, FBXO11, CREG1, DEDD, DVL1, TERF2IP, ZC3H7A, TYK2, CSNK1G2, SCARB1, E4F1, HSBP1, ZCCHC9, BCKDK, PRKD2, CENPB, FBXW7, ZNF688, UBE2D3, SIGIRR, IKBKE, MED25, ASB7, H3F3A, CRTC1, FLYWCH1, AHCTF1, ESRRA, NFKBIB, ZNF616, CDK3, PPP1R15A, AKT1S1, ARID4B, SETD1B, ERO1L, TCEANC2, MAP3K11, PSMB10, PRKCSH, ZNF358, ZNF493, PPM1A, MAPK8IP3, JRKL, AGPAT2, HIST1H1C, WASF2, C14orf169, RIN2, EED, ZNF579, SCAI, MYBL2, DDX20, CLN3, HIRA, ZC4H2, XPR1, PUF60, and HOXB2.

In a second aspect of the fifth example embodiment, the set of proteins is IRF3, ARL2BP, ZBTB17, and ATRX.

In a third aspect of the fifth example embodiment, the set of proteins is selected by cross-validation.

In a fourth aspect of the fifth example embodiment, the set of proteins consists of proteins having at least a pre-determined value of differential protein activity between responders and non-responders.

In a fifth aspect of the fifth example embodiment, the protein activity value is a normalized enrichment score.

In a sixth aspect of the fifth example embodiment, determining the plurality of protein activity values comprises applying VIPER algorithm to gene expression data of the subject.

In a seventh aspect of the fifth example embodiment, the trained classifier comprises a support vector machine, an artificial neural network, a random forest, a linear classifier, linear discriminant analysis, logistic regression, or ridge regression.

### Computer-Implemented Methods

In a sixth example embodiment, the present invention is a computer program product for identifying responders and non-responders, the computer program product comprising a computer readable storage medium having program instructions embodied therewith, the program instructions executable by a processor to cause the processor to perform a method comprising determining a plurality of protein activity values in a subject suffering from multiple myeloma (MM), each protein activity value corresponding to one of a set of proteins in the subject; providing the plurality of protein activity values to a trained classifier, the trained classifier being trained to differentiate between responders and non-responders to a therapy by a compound represented by structural formula (1); and obtaining from the classifier a classification of the subject as a responder or non-responder,

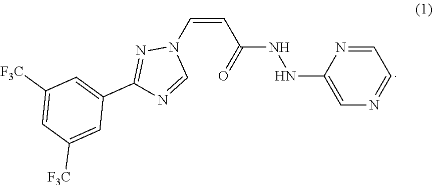 [SMILES: O=C(/C=C\n1cnc(-c2cc(C(F)(F)F)cc(C(F)(F)F)c2)n1)NNc1cnccn1]

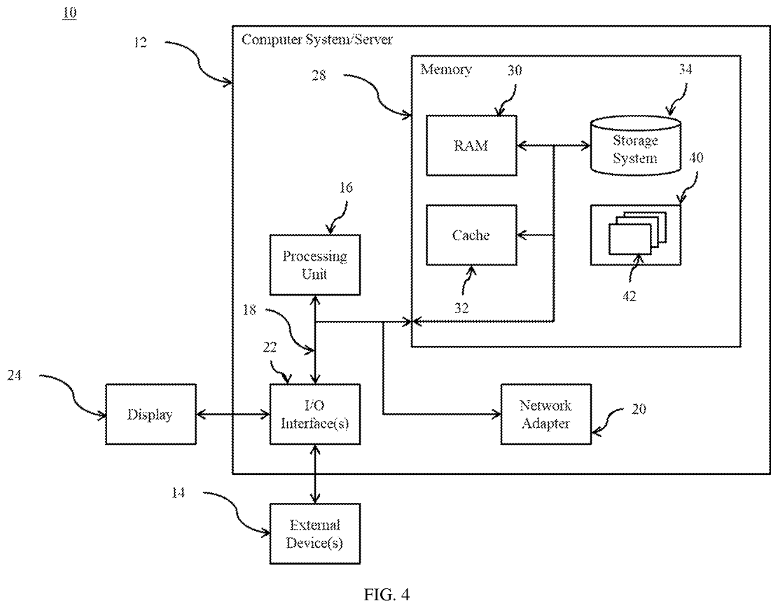

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the FIG. 4 illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXEMPLIFICATION

### Example 1: STORM Study

Selinexor 80 mg in combination with dexamethasone 20 mg administered using 2 dosing schedules was studied in patients with MM refractory to either 4 or 5 drugs (quad- and penta-refractory) in Part 1 of the phase 2 STORM (Selinexor Treatment Of Refractory Myeloma) study. Median overall response rate (ORR) was 21% in this heterogeneous population. Based on these findings, the activity of selinexor 80 mg administered twice-weekly was examined in a more uniform population in the pivotal STORM Part 2 study.

### Patients

Eligible patients had measurable MM by International Myeloma Working Group (IMWG) criteria (See, Durie B G, Harousseau J L, Miguel J S, et al. International uniform response criteria for multiple myeloma. Leukemia 2006;20:1467-73. Rajkumar S V, Harousseau J L, Durie B, et al. Consensus recommendations for the uniform reporting of clinical trials: report of the International Myeloma Workshop Consensus Panel. Blood 2011;117:4691-5); prior treatment with bortezomib, carfilzomib, lenalidomide, pomalidomide, daratumumab, glucocorticoids, and an alkylating agent; and had disease refractory to at least 1 IMiD (lenalidomide and pomalidomide), 1 PI (bortezomib or carfilzomib), daratumumab, glucocorticoids and to their last regimen. Refractory disease was defined as progression during or within 60 days after completion of therapy, or <25% response to therapy.19, 20 Eastern Cooperative Oncology Group performance status of 0-2, adequate hepatic function, renal function, and hematopoietic function were required. Systemic light chain amyloidosis, active central nervous system involvement, grade≥3 peripheral or grade≥2 painful neuropathy were exclusion criteria.

### Treatment

Oral selinexor 80 mg in combination with dexamethasone 20 mg was administered on days 1 and 3, weekly, in 4-week cycles until disease progression, death or discontinuation. A dose modification protocol was used for adverse event (AE) management. All patients were required to receive ondansetron 8 mg (or equivalent) prior to the first dose of study drug and 2-3 times daily as needed. Other antiemetics (olanzapine, NK-1R antagonists) were permitted for patients intolerant to or with persistent nausea. Supportive measures were provided at the discretion of the investigator and may have included intravenous fluids, hematopoietic growth factors, transfusions, and/or appetite stimulants (olanzapine, megesterol acetate).

### Endpoints and Assessments

The primary endpoint was Overall Response Rate (ORR) adjudicated by the appointed Independent Review Committee (IRC). Secondary endpoints included duration of response (DOR), clinical benefit rate (CBR), progression-free survival (PFS) and OS (Overall Survival). Disease-specific assessments were conducted at baseline, day 1 of each treatment cycle, and at the time of disease progression or suspected response. High-risk cytogenetics included del(17p), t(4;14), t(14;16), and gain(1q) chromosomal abnormalities by fluorescent in situ hybridization (FISH). Quality of life was assessed using the Functional Assessment of Cancer Therapy-Multiple Myeloma (FACT-MM) patient-reported outcome questionnaire. Safety and tolerability were assessed through history, physical exam, laboratory assessments and 12-lead electrocardiogram. Adverse events (AEs) were graded according to the NCI CTCAE v4.03.

### Statistical Analysis

The sample size was based on assumptions for penta-exposed, triple-class-refractory MM using a minimal threshold for ORR of 10%. For the primary efficacy analysis, a sample size of 122 patients allowed for a one-sided test at α=0.025 to detect an ORR of ≥20% against the threshold ORR of 10% with 90% power. The modified intention-to-treat (mITT) population was used for the primary efficacy analysis, comprised of all enrolled patients who met all eligibility criteria or received a waiver from the Sponsor to enroll in the trial and received at least 1 dose of selinexor plus dexamethasone. The safety population included all patients who received at least 1 dose of study drug. The primary analysis used a 2-sided, exact 95% confidence interval, calculated for the ORR among the mITT population, with statistical significance declared if the lower bound of this interval was >10%. Summary statistics were computed and displayed for each of the defined analysis populations and by each assessment timepoint. Summary statistics for continuous variables minimally included: n (number), mean, standard deviation (SD), minimum, median, and maximum. For categorical variables, frequencies and percentages are presented. For time-to-event variables, the Kaplan-Meier method was used for descriptive summaries.

### Pharmacodynamics and Response Predictor

Selinexor binding to XPO1 leads to rapid inactivation of nuclear export, XPO1 protein degradation, and induction of XPO1 mRNA transcription (without new protein production). XPO1 mRNA induction is therefore a pharmacodynamic marker in selinexor-treated patients.

RNA levels in CD138+ cells isolated from the pre-treatment bone marrow aspirate of patients in STORM Part 2 were used to populate the VIPER algorithm (See Example 2).

### Results

A total of 123 patients were enrolled, all of whom were included in the safety population. One patient did not meet full eligibility criteria (no prior carfilzomib); therefore 122 patients were included in the mITT population. Median age was 65.2 years, median duration of MM was 6.6 years, and 53% had high-risk cytogenetics.

All had progressive MM at time of enrollment and was typically rapidly progressive: 89 patients (73%) with available data had a median increase in disease burden of 22% (range, −42.8-1000) between screening and first day of therapy (median 12 days). Creatinine clearance was <60 mL/min in 39 patients (32%) and <40 mL/min in 14 (11.5%). Median number of therapies was 7 (range 3-18); 86 (70%) patients had prior daratumumab combined with other agents, 102 (83.6%) had prior stem cell transplantation, and 2 had prior chimeric antigen receptor T-cell (CAR-T) therapy. In the mITT population, all patients had penta-exposed MM refractory to at least 1 Proteosome Inhibitor (PI), 1 immunomodulatory agent (IMiD), and daratumumab as required by protocol. Sixty-eight percent (68%) were documented to have penta-refractory MM, ˜19% and 13% had MM not refractory to bortezomib or lenalidomide, respectively, and were included due to intolerance, or inability to document progression by IMWG criteria. Importantly, 95.9% had MM refractory to the most potent agent of each class: carfilzomib, pomalidomide, daratumumab.

### Treatment Duration and Doses

Of the 123 patients enrolled, 118 (95.9%) discontinued treatment, with disease progression (55.1%) and AEs (unrelated and related, 32.5%) the most common reasons. At the last date of follow up (17 Aug. 2018), 5 (4.1%) patients remained on treatment; 34 (27.6%) were off treatment and in long-term survival follow-up. The median duration of selinexor plus dexamethasone treatment was 9.0 weeks (range, 1-60).

### Efficacy

The ORR was 26.2% (95% CI, 18.7, 35.0), including 2 (1.6%) stringent complete responses, 6 (4.9%) very good partial responses, and 24 (19.7%) partial responses.

Both patients with relapse after CAR-T achieved a partial response. Minimal response was observed in 16 (13.1%) patients and 48 patients (39.3%) had stable disease, while 26 (21.3%) had progressive disease or whose disease was not evaluable. Median time to partial response or better was 4.1 weeks (range, 1-14 weeks). CBR (≥minimal response), was 39.3% (95% CI, 30.6, 48.6). The median DOR was 4.4 months (95% CI, 3.7, 10.8). PFS was 3.7 months (95% CI, 3.0, 5.3) and OS was 8.6 months (95% CI, 6.2, 11.3). In patients who achieved a partial or minimal response or better, median OS was 15.6 months.

In this pivotal trial, patients with penta-exposed, triple-class refractory MM treated with oral selinexor, a first-in-class XPO1 inhibitor, with dexamethasone twice-weekly, resulted in an ORR of 26.2%. Responses were rapid and deep, with 2 patients achieving stringent complete responses and 6 with very good partial responses. The observed efficacy was consistent across subgroups, including patients with high-risk cytogenetics (53% of the patients). While cross-trial comparisons are challenging and limited by differences in patient populations, inclusion/exclusion criteria, and overall study conduct, our results in penta-exposed, triple-class refractory MM compare favorably to those from other studies in refractory MM populations: ORR to carfilzomib was 18.9% in bortezomib-refractory disease and in the most comparable population (quad-refractory myeloma), the ORR for daratumumab was 21.2% in the pivotal phase 2 study, being somewhat higher at 36% in the expansion cohort (n=15) of the phase 1 monotherapy study.

### Example 2: Biomarkers for Selinexor Response in MM

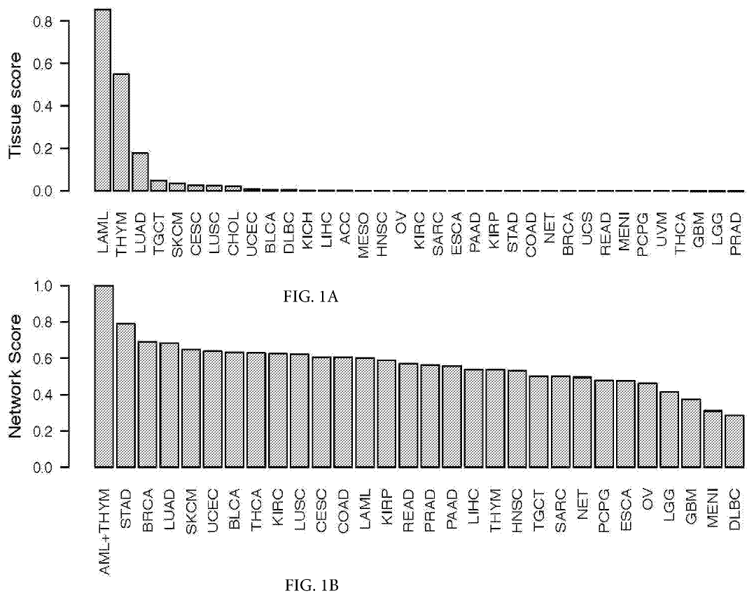

The transcriptome for 2 separate batches of pre-treatment biopsies, from patients enrolled in the STORM (Parts 1 and 2) trial, was profiled by RNA-Seq. The activity of 6,204 regulatory proteins was inferred by metaVIPER, using acute myeloid leukemia (AML) and thymoma context-specific model of transcriptional regulation (interactomes), which were selected among 29 available interactomes based on tissue lineage supervised classification and network representation analysis (FIG. 1).

According to FIG. 1A and FIG. 1B, identification of the most appropriate tissue context-specific interactomes for MM was based on the likelihood predicted by a tissue-type classifier based on gene expression (FIG. 1A), and the Network Score, representing how well each evaluated interactome can explain the transcriptional state of the MM samples. As shown in FIG. 1B, AML+THYM represents the integration of acute myeloid leukemia and thymoma interactomes by metaVIPER.

Unlike raw gene expression profiles, VIPER-inferred protein activity is extremely reproducible, and this methodology (DarwinOncoTarget algorithm) has been approved by the NYS Department of Health CLIA/CLEP Validation Unit for Molecular and Cellular Tumor Markers for Oncology.

A training set comprising 42 samples from patients enrolled in STORM part 2 was assembled. Responders included Complete Response (sCR), Very Good Partial Response (VGPR), and Partial Response (PR) with DOCB>36 days. Non-responders included Progressive Disease (PD) and Stable Disease (SD) samples treated longer than 30 days.

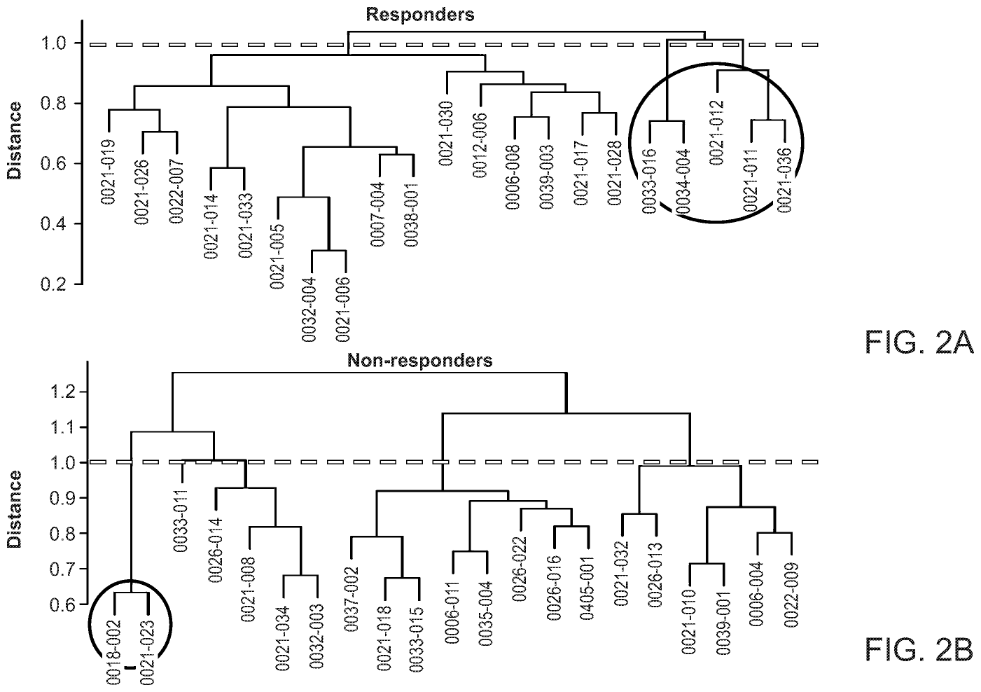

The homogeneity of the regulatory mechanisms associated with selinexor responder and non-responder phenotypic states was inspected. For this, regulatory protein activity signatures for each responder sample were obtained by comparison against the pool of selinexor non-responders (21 samples). Similarly, regulatory protein activity signatures were obtained for each non-responder sample by comparison against the pool of selinexor responders (21 samples). To evaluate the homogeneity of these signatures, unsupervised hierarchical cluster analysis was performed. This analysis indicated that responder and non-responder protein activity signatures are heterogeneous, and they potentially represent several distinct mechanisms of response, as well as three distinct mechanisms of resistance to selinexor treatment (FIG. 2).

FIG. 2A and FIG. 2B represents dendrograms showing the unsupervised clustering of the samples based on their similarity in MR signatures for patients that responded (FIG. 2A) and did not respond (FIG. 2B) to selinexor.

Since at least 3 samples per mechanistic cluster are required for proper analysis, 5 samples from among the responders and 2 samples from the non-responders (highlighted by circles in FIG. 2), which were different from the rest of the samples in the same class, were removed from the training set. This left 16 responders and 19 non-responders for further analysis.

Based on the remaining 35 samples, five classifiers were trained—including Linear Discriminant Analysis (LDA), Logistic regression, Neural Network, Random Forest, and Ridge regression methods. Using VIPER, a detailed inspection of the MR protein activity signatures characteristic of each responder and non-responder sample indicated heterogeneity in the regulatory mechanisms leading to a selinexor resistant of susceptible tumor phenotype. The results from this analysis were useful for identifying distinct mechanisms leading to responder and non-responder phenotypic states and the samples representing them.

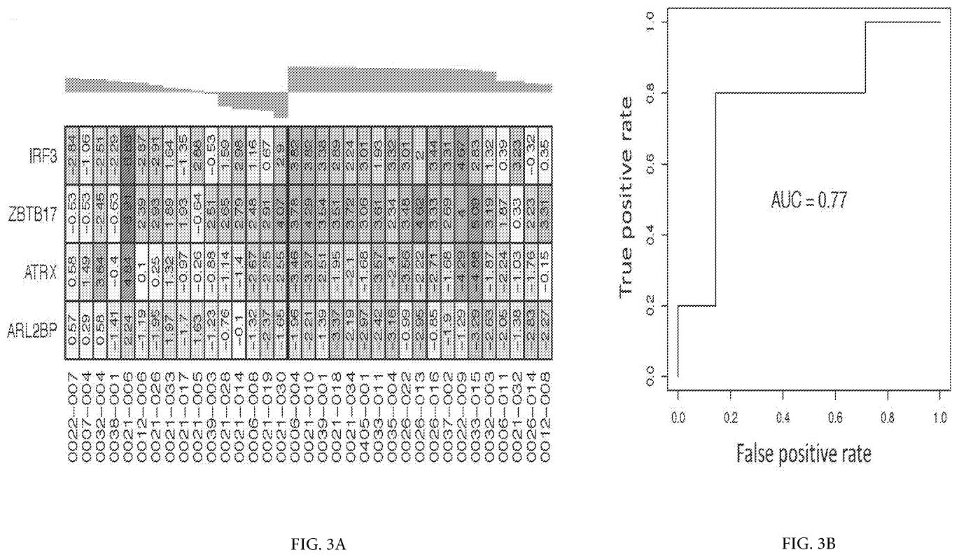

Leave-one-out cross-validation (LOOCV) analysis achieved best performance using the following top four Master Regulator proteins IRF3, ARL2BP, ZBTB17, and ATRX (FIG. 3A), with AUC scores equal to 0.862, 0.862, 0.767, 0.697, and 0.806, respectively (FIG. 3B).

FIG. 3A and FIG. 3B present data that demonstrate clinical benefits of the biomarkers for MM patients being treated with solinexor. FIG. 3A is heatmap showing the relative protein activity for the 4 MR proteins. The bar above the heatmap shows the silhouette score for each sample, computed based on Euclidean distance, with Responder and Non-responder samples shown on the left and on the right, respectively. The values inside each cell in the heatmap shows the relative protein activity for each MR protein in each sample. FIG. 3B is a plot showing the evaluation of the biomarker performance in an independent sample set. Shown is the ROC analysis and estimated AUC.

The performance of the best classifier (LDA) as a biomarker of clinical benefit was then tested on an independent set of samples, which were profiled as a separate batch, comprising 12 samples from MM patients enrolled in the STORM (part 1 and 2) trial. The analysis confirmed the value of the biomarker as an effective classification metrics, with a ROC AUC=0.77. Within the limitations imposed by a small testing cohort of only 12 samples, the selinexor CB biomarker correctly identified 4 out of 5 (80%) responder patients at a false positive rate below 20% and misclassified only 1 out of 7 non-responder patients, yielding a prediction accuracy of 83%, which, nevertheless, showed a very high overall survival of 511 days.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of treating a patient suffering from multiple myeloma, comprising:
determining a plurality of protein activity values in a subject suffering from multiple myeloma (MM), each protein activity value corresponding to one of a set of proteins in the subject;
determining a classification of the subject as a responder or non-responder to a therapy by a compound represented by structural formula (1); and
administering a therapeutically effective amount of the compound represented by structural formula (1) or a pharmaceutically acceptable salt thereof 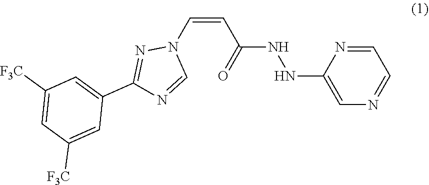 [SMILES: O=C(/C=C\n1cnc(-c2cc(C(F)(F)F)cc(C(F)(F)F)c2)n1)NNc1cnccn1]
to the subject determined to be responder.

2. A method of treating a subject suffering from multiple myeloma, comprising:
administering a therapeutically effective amount of a compound represented by structural formula (1) or a pharmaceutically acceptable salt thereof 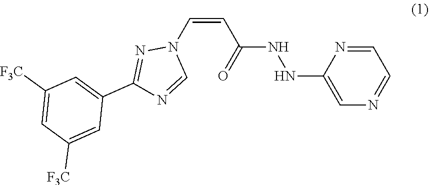 [SMILES: O=C(/C=C\n1cnc(-c2cc(C(F)(F)F)cc(C(F)(F)F)c2)n1)NNc1cnccn1]
to the subject suffering from multiple myeloma,
wherein the subject is determined to be a responder to a therapy by the compound represented by structural formula (1) based on a plurality of protein activity values in the subject, each protein activity value corresponding to one of a set of proteins in the subject.

3. A method of treating a subject suffering from multiple myeloma, comprising:
selecting the subject suffering from multiple myeloma only if the subject is determined to be a responder to a therapy by a compound represented by structural formula (1) based on a plurality of protein activity values in the subject, each protein activity value corresponding to one of a set of proteins in the subject; and
administering to the selected subject a therapeutically effective amount of the compound represented by structural formula (1) or a pharmaceutically acceptable salt thereof

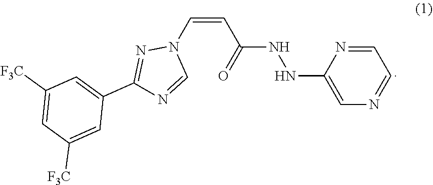 [SMILES: O=C(/C=C\n1cnc(-c2cc(C(F)(F)F)cc(C(F)(F)F)c2)n1)NNc1cnccn1]

4. A method of treating a subject suffering from multiple myeloma, comprising:
receiving information of a plurality of protein activity values in a subject suffering from multiple myeloma (MM), each protein activity value corresponding to one of a set of proteins in the subject; and
administering to the subject a therapeutically effective amount of a compound represented by structural formula (1) or a pharmaceutically acceptable salt thereof 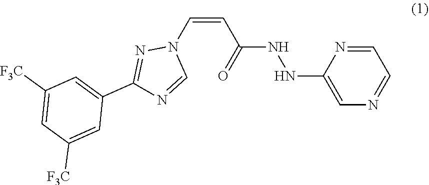 [SMILES: O=C(/C=C\n1cnc(-c2cc(C(F)(F)F)cc(C(F)(F)F)c2)n1)NNc1cnccn1]
only if the subject is determined to be a responder to a therapy by the compound represented by structural formula (1) based on said plurality of protein activity values.

5. The method of any one of claims 1-4, wherein the set of proteins is selected from IRF3, ARL2BP, ZBTB17, ATRX, MPP7, TDP2, ATF1, FBXW11, C1D, PKD1, GDI2, SUPT5H, SHOC2, RBCK1, ZNF598, ZNF697, PRKACB, SIRT7, RPS6KB1, RAB1A, ZNF575, MBTD1, ZNF24, TBL3, MYBBP1A, CELSR1, SETD1A, TP53, CASP8AP2, ZNF28, STK11, SMARCA4, SIRT1, ZNF324B, ZNF532, MBD3, ZFYVE16, CSDE1, IFT27, PER1, FBXO11, CREG1, DEDD, DVL1, TERF2IP, ZC3H7A, TYK2, CSNK1G2, SCARB1, E4F1, HSBP1, ZCCHC9, BCKDK, PRKD2, CENPB, FBXW7, ZNF688, UBE2D3, SIGIRR, IKBKE, MED25, ASB7, H3F3A, CRTC1, FLYWCH1, AHCTF1, ESRRA, NFKBIB, ZNF616, CDK3, PPP1R15A, AKT1S1, ARID4B, SETD1B, ERO1L, TCEANC2, MAP3K11, PSMB10, PRKCSH, ZNF358, ZNF493, PPM1A, MAPK8IP3, JRKL, AGPAT2, HIST1H1C, WASF2, C14orf169, RIN2, EED, ZNF579, SCAI, MYBL2, DDX20, CLN3, HIRA, ZC4H2, XPR1, PUF60, and HOXB2.

6. The method of any one of claims 1-5, wherein the set of proteins is IRF3, ARL2BP, ZBTB17, and ATRX.

7. The method of any one of claims 1-6, further comprising:
collecting a bone marrow sample from the subject;
separating CD131+ cells in the bone marrow sample;
identifying the activity pattern of the MR proteins in the CD131+ cells.

8. The method of any one of claims 1-7, wherein the multiple myeloma is a relapsed or refractory multiple myeloma.

9. The method of any one of claims 1-8, wherein the subject has received from 1 to 7 prior therapies.

10. The method of claim 9, wherein the subject has received at least two prior therapies.

11. The method of claim 9, wherein the subject has received at least three prior therapies.

12. The method of claim 9, wherein the subject has received at least four prior therapies.

13. The method of any one of claims 1-12, wherein the subject is a human.

14. The method of claim 13, wherein the human is an adult.

15. The method of any one of claim 1-14, wherein the compound represented by formula (1) is administered orally.

16. The method of claim 15, wherein the multiple myeloma is refractory to at least two proteasome inhibitors, at least two immunomodulatory agents, and an anti-CD38 monoclonal antibody.

17. The method of any one of claims 1-16 further comprising administering at least one additional therapeutic agent.

18. The method of claim 17, wherein the additional therapeutic agent is dexamethasone.

19. The method of claim 18, wherein the dexamethasone is orally administered at an amount of 20 mg/day.

20. The method of any one of claim 18 or 19, further comprising administering bortezomib.

21. The method of any one of claims 1-7, wherein the multiple myeloma is relapsed or refractory multiple myeloma, the subject is an adult human who has received at least four prior therapies and the multiple myeloma is refractory to at least two proteasome inhibitors, at least two immunomodulatory agents and an anti-CD38 monoclonal antibody.

22. The method of claim 21, wherein the compound of formula (1) is administered at 80 mg/per day on days 1 and 3 of each week of treatment.

23. The method of claim 22, wherein an additional therapeutic agent is administered.

24. The method of claim 23, wherein the additional therapeutic agent is dexamethasone.

25. The method of claim 24, wherein the dexamethasone is administered at 20 mg/day on days 1 and 3 of each week of treatment.

26. The method of any one of claims 1-7, wherein the multiple myeloma is relapsed or refractory multiple myeloma, the subject is an adult human who has received from 1 to 3 prior therapies.

27. The method of claim 26, wherein the compound of formula (1) is administered at 100 mg once a week.

28. The method of claim 26 or claim 27, wherein at least one additional therapeutic agent is administered.

29. The method of claim 28, wherein the additional therapeutic agents are bortezomib administered at 1.3 mg/m2 once a week and dexamethasone administered twice a week at 20 mg per administration.

30. A method of identifying a subject as a responder or a non-responder, comprising:
determining a plurality of protein activity values in a subject suffering from multiple myeloma (MM), each protein activity value corresponding to one of a set of proteins in the subject;
providing the plurality of protein activity values to a trained classifier, the trained classifier being trained to differentiate between responders and non-responders to a therapy by a compound represented by structural formula (1); and
obtaining from the classifier a classification of the subject as a responder or non-responder,

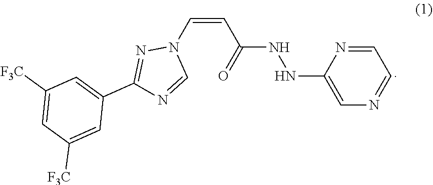 [SMILES: O=C(/C=C\n1cnc(-c2cc(C(F)(F)F)cc(C(F)(F)F)c2)n1)NNc1cnccn1]

31. The method of claim 30, wherein the set of proteins is selected from IRF3, ARL2BP, ZBTB17, ATRX, MPP7, TDP2, ATF1, FBXW11, C1D, PKD1, GDI2, SUPT5H, SHOC2, RBCK1, ZNF598, ZNF697, PRKACB, SIRT7, RPS6KB1, RAB1A, ZNF575, MBTD1, ZNF24, TBL3, MYBBP1A, CELSR1, SETD1A, TP53, CASP8AP2, ZNF28, STK11, SMARCA4, SIRT1, ZNF324B, ZNF532, MBD3, ZFYVE16, CSDE1, IFT27, PER1, FBXO11, CREG1, DEDD, DVL1, TERF2IP, ZC3H7A, TYK2, CSNK1G2, SCARB1, E4F1, HSBP1, ZCCHC9, BCKDK, PRKD2, CENPB, FBXW7, ZNF688, UBE2D3, SIGIRR, IKBKE, MED25, ASB7, H3F3A, CRTC1, FLYWCH1, AHCTF1, ESRRA, NFKBIB, ZNF616, CDK3, PPP1R15A, AKT1S1, ARID4B, SETD1B, ERO1L, TCEANC2, MAP3K11, PSMB10, PRKCSH, ZNF358, ZNF493, PPM1A, MAPK8IP3, JRKL, AGPAT2, HIST1H1C, WASF2, C14orf169, RIN2, EED, ZNF579, SCAI, MYBL2, DDX20, CLN3, HIRA, ZC4H2, XPR1, PUF60, and HOXB2.

32. The method of claim 30, wherein the set of proteins is IRF3, ARL2BP, ZBTB17, and ATRX.

33. The method of any one of claims 30-32, wherein the set of proteins is selected by cross-validation.

34. The method of any one of claims 30-33, wherein the set of proteins consists of proteins having at least a pre-determined value of differential protein activity between responders and non-responders.

35. The method of any one of claims 30-34, wherein the protein activity value is a normalized enrichment score.

36. The method of any one of claims 30-35, wherein determining the plurality of protein activity values comprises applying VIPER algorithm to gene expression data of the subject.

37. The method of any one of claims 30-36, wherein the trained classifier comprises a support vector machine, an artificial neural network, a random forest, a linear classifier, linear discriminant analysis, logistic regression, or ridge regression.

38. A computer program product for identifying responders and non-responders, the computer program product comprising a computer readable storage medium having program instructions embodied therewith, the program instructions executable by a processor to cause the processor to perform a method comprising:
determining a plurality of protein activity values in a subject suffering from multiple myeloma (MM), each protein activity value corresponding to one of a set of proteins in the subject;
providing the plurality of protein activity values to a trained classifier, the trained classifier being trained to differentiate between responders and non-responders to a therapy by a compound represented by structural formula (1) or a pharmaceutically acceptable salt thereof 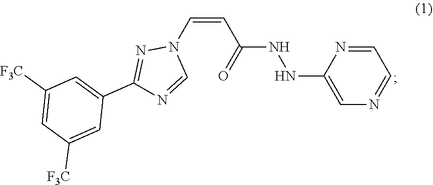 [SMILES: O=C(/C=C\n1cnc(-c2cc(C(F)(F)F)cc(C(F)(F)F)c2)n1)NNc1cnccn1]
and
obtaining from the classifier a classification of the subject as a responder or non-responder.
